(19) United States
(12) Patent Application Publication
(10) Pub. No.: US 2009/0110755 A1
(43) Pub. Date: Apr. 30, 2009

(54) Compositions and Methods for Preventing Migraines

(76) Inventor: Alexander Mauskop, Larchmont, NY (US)

Correspondence Address: MORGAN, LEWIS & BOCKIUS LLP, 1701 MARKET STREET, PHILADELPHIA, PA, 19103-2921, US

(21) Appl. No.: 12/260,482

(22) Filed: Oct. 29, 2008

Related U.S. Application Data
(60) Provisional application No. 60/983,790, filed on Oct. 30, 2007.

(51) Int. Cl. A61K 33/08; A61K 33/06; A61K 31/215; A61K 31/40; A61K 31/5415; A61K 31/407; A61K 31/404; A61K 31/415; A61K 31/42; A61K 31/421; A61K 31/444; A61P 9/00; A61P 29/00

(52) U.S. Cl. 424/692; 424/682; 514/547; 514/546; 514/423; 514/226.5; 514/413; 514/411; 514/415; 514/406; 514/378; 514/374; 514/334

(57) ABSTRACT

The present invention relates to compositions and methods for preventing migraines. The migraine prophylaxis composition is a single dosage form comprising at least one form of elemental magnesium and at least one NSAID. The prophylaxis composition, comprising at least one form of elemental magnesium and a therapeutically effective concentration of acetylsalicylic acid may also provide cardiac protection and prevent cardiovascular diseases.

### STATEMENT OF RELATED APPLICATIONS

This application claims priority to and the benefit of U.S. Patent Application Number 60/9835,790 filed Oct. 30, 2007, which is herein incorporated by reference in its entirety.

### BACKGROUND

Migraines are characterized by pulsating head pain that is episodic, unilateral or bilateral, lasting from about 4 to 72 hours and often associated with nausea, vomiting and hypersensitivity to light and or sound. National population statistics indicate that 21 million women and 7 million men suffer from severe migraine headaches. The precise etiology of migraines remains elusive.

Only about one-third of all migraine patients are fully satisfied with treatments currently available because these are generally no more than sporadically effective and are associated with significant side effects. Reported significant side effects include fainting, weight gain, shortness of breath, constipation, fatigue, memory impairment, insomnia, nausea, asthenia, drowsiness, and dizziness. In addition, some migraine treatments are potentially addictive with well-documented withdrawal symptoms.

Considering the contraindications and risk of side effects, an improved migraine drug formulation is needed for millions of migraine sufferers.

### SUMMARY OF THE INVENTION

According to some embodiments, a migraine prophylaxis composition includes at least one form of elemental magnesium and at least one NSAID, wherein said prophylaxis composition is a single dosage form.

According to some embodiments, a method of preventing migraines in a patient in need thereof includes administration of a migraine prophylaxis composition, wherein the prophylaxis composition is a single dosage form comprising at least one form of elemental magnesium and at least one NSAID.

According to some embodiments, a method of providing cardiac protection and preventing cardiovascular disease in a patient in need thereof includes administration of a prophylaxis composition, wherein the prophylaxis composition is a single dosage form comprising at least one form of elemental magnesium and a therapeutically effective concentration of acetylsalicylic acid.

### DETAILED DESCRIPTION OF THE INVENTION

As used throughout, ranges are used as shorthand for describing each and every value that is within the range. Any value within the range can be selected as the terminus of the range.

The term “prophylaxis composition” as used herein shall include any drug composition containing at least two therapeutically active components of which at least one is a form of elemental magnesium and at least one is a non-steroidal anti-inflammatory drug (NSAID).

The term “elemental magnesium” as used herein shall include any chemical composition that provides a therapeutically effective concentration of ionized Mg²⁺ to the patient to prevent migraine headache.

The term “NSAID” as used herein refers to any compound acting as a non-steroidal anti-inflammatory agent identifiable as such by one of ordinary skill in the art that provides a therapeutically effective concentration of the active ingredient to the patient to prevent migraine headache.

The term “single dosage form” as used herein shall include any single drug administration entity combining at least one form of elemental magnesium and at least one NSAID into a prophylaxis composition. In some embodiments, the single dosage form is a single tablet, capsule, or liquid.

The term “therapeutically effective concentration” as used herein shall include any drug dosage that provides the specific pharmacological response.

In some embodiments, a migraine prophylaxis composition of the present invention includes at least one form of elemental magnesium and at least one NSAID, wherein said prophylaxis composition is a single dosage form. In some embodiments, a migraine prophylaxis composition of the present invention including at least one form of elemental magnesium and at least one NSAID may be administered in a single dosage form to a patient in need thereof to prevent onset of migraine headache. In some embodiments, a migraine prophylaxis composition of the present invention including at least one form of elemental magnesium and at least one NSAID may be administered in a single dosage form to a patient in need thereof to treat arthritis and other inflammatory conditions. In some embodiments, a prophylaxis composition of the present invention including at least one form of elemental magnesian and a therapeutically effective concentration of acetylsalicylic acid may be administered in a single dosage form to a patient in need thereof to provide cardiac protection and prevent cardiovascular disease.

In some embodiments, a migraine prophylaxis composition of the present invention including at least one form of elemental magnesium and at least one NSAID produces surprising results. Without being bound by any one particular theory, in one embodiment, the two active ingredients work through different mechanisms to inhibit the enzyme cyclooxygenase synthase (COX) resulting in surprising efficacy. Pharmacological inhibition of COX prevents the conversion of arachidonic acid into prostanoids involved in inflammation and pain. Magnesium inhibits the synthesis of the COX-2 isoenzyme while NSAIDs inhibit the action of the COX-1 and or COX-2 isoenzymes.

Similarly, magnesium and acetylsalicylic acid, and to a lesser extent other NSAIDs, has been found to inhibit platelet aggregation through different mechanisms and this may also account for their synergistic effect in the prevention of migraines and cardiovascular disease. Case control studies suggest a definite relation between platelet aggregation and vascular disease and both magnesium and acetylsalicylic acid have been shown to reduce the generation of prothrombotic eicosanoids that play an important role in the pathogenesis of hypertension, athersclerosis and ischemic heart disease.

Another unexpected benefit of combining NSAIDS with magnesium is the reduction of the risk of cardiovascular complications due to COX-2 and the side effects of certain NSAIDs. NSAIDs other than acetylsalicylic acid have been shown to block vasodilation produced by intracellular magnesium, potentially leading to cardiovascular complications. The introduction of additional magnesium may reverse the vasodilation block, as well as enhance absorption of an NSAID and reduce another NSAID side effect by protecting the stomach.

Magnesium

Magnesium deficiency may be involved in both vascular and neurological aspects (e.g., migraine aura, pain, nausea, etc.) of the development of migraines. Changes in magnesium levels have effects on the functions of serotonin and NMDA receptors, nitric oxide production, catecholamine production and activity and on more than 325 enzymatic processes in the body. Ionized Mg²⁺ levels are known to affect entry of Ca²⁺, intracellular Ca²⁺ levels and its release from sarcoplasmic and endoplasmic reticulum membranes in vascular muscle and endothelial cells which in turn control vascular tone and vascular reactivity to endogenous hormones and neurotransmitters. Such vascular effects could play a role in migraine pathogenesis. Drugs that have been successfully utilized for the treatment or prevention of migraine attacks act on the brain or cerebral blood vessels. Unfortunately, such drugs also cause significant side effects linked to excessive vasoactivity in regions of the body not involved in the pathogenesis of migraine.

The present invention explores in part the possibility that magnesium deficiency could also be involved in the development of migraines and is related to the combination of at least one form of elemental magnesium and at least one non-steroidal anti-inflammatory drug (NSAID) in a single oral pharmaceutical dosage form. One embodiment of the inventive formulations and methods described herein prevent the onset of migraines without significant side effects, thereby circumventing both the debilitating symptoms of migraine headaches as well as the side effects of conventional treatments. In addition, one embodiment of the present invention is not contraindicated in patients with coexisting cardiovascular disease or risk of cardiovascular disease. In some embodiments, the prophylaxis composition provides cardiac protection and prevents cardiovascular disease.

Cerebral blood vessels are more sensitive to magnesium than any other type of vascular muscle cells. Magnesium-deficiency results in contraction and potentiation of vasoconstrictors. Deficiency of Mg²⁺ in neuronal and vascular tissue could be expected to result in elevation in levels of catecholamines, serotonin, prostaglandins, and substance P—vasoactive substances long implicated in the etiology of migraine. In addition, serotonin is known to be released from platelets during a migraine attack, and to be a potent cerebral vasoconstrictor and promote nausea and vomiting. Some prostanoids, e.g., PGE₁, are known to provoke pulsating headaches and additional migraine-like symptoms as well as potentiate vasoconstrictor hormones acting on blood vessels. Headache pain is often attributed to the action of substance P on sensory fibers. It is of some interest to note that a magnesium-deficiency induced state has recently been shown experimentally to result in significant generation and release of substance P into the blood stream.

Magnesium also seems to possess unique weak calcium channel-blocking abilities. It can act to regulate the entry of Ca²⁺ into smooth muscle and endothelial cells, and regulate the intracellular release and binding of Ca²⁺ by acting on potential-operated, receptor-operated and leak-operated Ca²⁺ channels in these cells. This may also help to explain why calcium channel blockers (CCB), unlike Mg²⁺, require a relatively long period of therapy (e.g., two-to-five months) to achieve reasonable therapeutic effects in migraine sufferers. CCB could not be expected to act on leak-operated Ca²⁺ channels and only certain ones act on receptor-operated Ca²⁺ channels, making Mg²⁺ a potentially ideal therapeutic agent for migraines. Furthermore, P-nuclear magnetic resonance spectroscopy showed low brain Mg²⁺ in a limited number of subjects during and between migraine attacks. A low Mg²⁺ level is known to facilitate the development of spreading depression of Leao in animal experiments. This phenomenon is thought to underlie the phenomenon of migraine aura.

Without being bound by any one particular theory, it is hypothesized that an effective concentration of elemental magnesium in the present invention produces its antinociceptive effect and prophylactic effect in part by antagonizing Ca²⁺-dependent nitric oxide release by vascular endothelial cells. Nitric oxide (NO) is a potent vasoconstrictor and also has a stimulatory effect on COX-2 mRNA expression. In one embodiment, the inhibitory effect of magnesium is most likely due to divalent cation competitive antagonism of intracellular calcium. By inhibiting NO synthase, magnesium effectively interferes with the biosynthesis of COX-2 by inhibiting COX-2 mRNA synthesis. The synergistic antinociceptive and prophylactic interaction of magnesium and an NSAID consequently produces a surprising and superior therapeutic advantage by blocking both the synthesis and the enzymatic action of COX.

In one embodiment of the present invention, a prophylaxis composition provides in part a therapeutically effective concentration of ionized Mg²⁺ to the patient to prevent migraine headache. In some embodiments, the concentration of elemental magnesium is at least 150 mg.

In some embodiments, the elemental magnesium is a magnesium salt or a mixture of magnesium salts. In some embodiments, at least one magnesium salt includes at least one of: magnesium oxide, magnesium aspartate, magnesium diglycinate, magnesium lactate, magnesium gluconate, magnesium pidolate, magnesium sulfate, trimagnesium dicitrate, magnesium pyrrolidone carboxylic acid, magnesium citrate, magnesium chloride, and combinations thereof.

NSAID

Non-steroidal anti-inflammatory drugs (NSAIDs) exert most of their anti-inflammatory, analgesic and antipyretic activity through inhibition of prostaglandin G/H synthase, also known as cyclooxygenase (COX). In some forms of migraine, certain patients have partial relief with the use of NSAIDs, but these agents, when taken alone, are rarely effective in providing complete and rapid relief of all the symptoms of migraine, especially when the symptoms of the attack already include nausea or vomiting. However, in one embodiment of the present invention, a therapeutically effective concentration of at least one NSAID may act as a migraine prophylaxis through the drug's known analgesic action, and or by reducing neurogenic and vascular inflammation or by other mechanisms such as platelet inhibition, for example.

In one embodiment, a prophylaxis composition provides in part a therapeutically effective concentration of at least one NSAID to the patient to prevent migraine headaches.

In some embodiments, the NSAID includes at least one of: meloxicam, diclofenac, ketorolac, etodolac, diflunisal, tolmetin, nabumeton, mefenamic acid, meclofenamate, sulindac, indomethacin, celecoxib, valdecoxib, rofecoxib, lumiracoxib, oxaprozin, piroxicam, acetylsalicylic acid, etoricoxib, naproxen, choline magnesium trisalicylate, ketoprofen, flurbiprofen, fenoprofen, ibuprofen, and combinations thereof.

In some embodiments, the migraine prophylaxis composition includes about 7.5 mg to about 15 mg meloxicam. In some embodiments, the migraine prophylaxis composition includes about 50 mg to about 75 mg diclofenac. In some embodiments, the migraine prophylaxis composition includes about 50 mg to about 75 mg indomethacin. In some embodiments, the migraine prophylaxis composition includes about 200 mg to about 400 mg celecoxib. In some embodiments, the migraine prophylaxis composition comprises about 10 mg to about 20 mg piroxicam.

In some embodiments, the migraine prophylaxis composition includes a therapeutically effective concentration of NSAID to provide cardiac protection and prevent cardiovascular disease. In some embodiments, the migraine prophylaxis composition includes a therapeutically effective concentration of acetylsalicylic acid.

Example Preparation of a Prophylaxis Composition

A prophylaxis composition as described above may be intended for oral use and may be prepared according to any method known in the art. In one embodiment, prophylaxis compositions or the present invention may be employed in admixtures with conventional excipients, e.g., pharmaceutically acceptable organic or inorganic carrier substances suitable for oral administration. Suitable pharmaceutically acceptable carrier substances include, but are not limited to, at least one of: water, salt solutions, alcohols, gum arabic, vegetable oils, benzyl alcohols, polyethylene glycols, gelate, carbohydrates such as lactose, amylose or starch, magnesium stearate talc, silicic acid, vicious paraffin, perfume oil, fatty acid monoglycerides and diglycerides, pentaerythritol fatty acid esters, hydroxymethylcellulose, polyvinylpyrrolidone, and the like. The pharmaceutical preparations may be sterilized and if desired mixed with auxiliary agents (e.g., lubricants, preservatives, stabilizers, wetting agents, emulsifiers, salts for influencing osmotic pressure buffers, coloring, flavoring and/or aromatic substances and the like). They may also be combined where desired with other active ingredients.

In some embodiments, the single dosage form is an oral tablet comprising the prophylaxis composition and one or more pharmaceutically acceptable excipients. Such excipients include, for example, an inert diluent such as lactose, granulating and disintegrating agents such as cornstarch, binding agents such as starch, and lubricating agents such as magnesium stearate. The tablets may be uncoated or coated by known techniques for elegance or to delay the release of the active ingredients.

In some embodiments, the single dosage form is a hard gelatin capsule. In some embodiments, the primary dosage hard gelatin capsule includes the prophylaxis composition and one or more pharmaceutically acceptable substrates. Example substrates include, for instance, inert beads, particles, granules, or pellets. In some embodiments where the substrates comprise inert pharmaceutically acceptable beads, the prophylaxis composition may be mixed with further ingredients prior to being coated onto the beads. Ingredients include, but are not limited to, at least one of: binders, surfactants, fillers, disintegrating agents, alkaline additives or other pharmaceutically acceptable ingredients, alone or in mixtures. Binders may include, for example, at least one of: celluloses such as hydroxypropyl methylcellulose, hydroxypropyl cellulose and carboxymethylcellulose sodium, polyvinyl pyrrolidone, sugars, starches, other pharmaceutically acceptable substances with cohesive properties, and combinations thereof. Suitable surfactants include, for example, pharmaceutically acceptable non-ionic or ionic surfactants such as sodium lauryl sulfate. In some embodiments, the single dosage form is a soft gelatin capsule comprising a suspension of the prophylaxis composition in a pharmaceutically acceptable vegetable or mineral oil.

In some embodiments, the single dosage form is a liquid. In some embodiments, the liquid is an aqueous suspension. In some embodiments, the primary aqueous suspension includes the prophylaxis composition and one or more excipients suitable as suspending agents such as pharmaceutically acceptable synthetic gums such as hydroxypropyl methylcellulose or natural gums. In some embodiments, the liquid is an oily suspension. In some embodiments, the primary oily suspension is formulated by suspending the prophylaxis composition in a vegetable oil or mineral oil. The oily suspensions may contain a thickening agent such as beeswax or cetyl alcohol. In some embodiments, a syrup, elixir, or the like can be used wherein the sweetened vehicle is employed.

In some embodiments, the single dosage form may contain the elemental magnesium and or the NSAID in immediate or sustained release form.

In some embodiments, wherein the rationale for administration of the claimed invention is prophylaxis, it is contemplated that in some preferred embodiments, the prophylaxis composition is incorporated into the single dosage form in a sustained release state. In some embodiments, the prophylaxis composition may be formulated as a sustained release oral formulation in any suitable tablet, coated tablet or multi-particulate formulation known to those skilled in the art.

Use of a Prophylaxis Composition

A prophylaxis composition as described above may be orally administered in a single dosage form. Administration of a single dosage form will result in blood levels of the elemental magnesium and NSAID required to produce a prophylactic effect.

In some embodiments, administration of the prophylaxis composition prevents migraine headache. In some embodiments, administration of the prophylaxis composition also provides cardiac protection and prevents cardiovascular disease.

In some embodiments, the prophylaxis composition is administered on a daily, ongoing basis. In some embodiments, the prophylaxis composition is administered most preferably once a day, though it may also be administered twice a day.

Although the foregoing description is directed to the preferred embodiments of the invention, it is noted that other variations and modifications in the details, materials, steps and arrangement of parts, which have been herein described and illustrated in order to explain the nature of the preferred embodiments of the invention, will be apparent to those skilled in the art, and may be made without departing from the spirit or scope of the invention.

What is claimed is:

1. A migraine prophylaxis composition comprising:
(i) at least one form of elemental magnesium; and
(ii) at least one NSAID;
wherein said prophylaxis composition is a single dosage form.

2. The prophylaxis composition of claim 1, wherein the concentration of elemental magnesium is at least 150 mg.

3. The prophylaxis composition of claim 1, wherein the elemental magnesium is a magnesium salt.

4. The prophylaxis composition of claim 3, wherein the magnesium salt is selected from the group consisting of: magnesium oxide, magnesium aspartate, magnesium diglycinate, magnesium lactate, magnesium gluconate, magnesium pidolate, magnesium sulfate, trimagnesium dicitrate, magnesium pyrrolidone carboxylic acid, magnesium citrate and magnesium chloride.

5. The prophylaxis composition of claim 1, wherein the NSAID is selected from the group consisting of meloxicam, diclofenac, ketorolac, etodolac, diflunisal, tolmetin, nabumeton, mefenamic acid, meclofenamate, sulindac, indomethacin, celecoxib, valdecoxib, rofecoxib, lumiracoxib, oxaprozin, piroxicam, acetylsalicylic acid, etoricoxib, naproxen, choline magnesium trisalicylate, ketoprofen, flurbiprofen, fenoprofen and ibuprofen.

6. The prophylaxis composition of claim 5, wherein said composition comprises about 7.5 to 15 mg meloxicam.

7. The prophylaxis composition of claim 5, wherein said composition comprises about 50 to 75 mg diclofenac.

8. The prophylaxis composition of claim 5, wherein said composition comprises about 50 to 75 mg indomethacin.

9. The prophylaxis composition of claim 5, wherein said composition comprises about 200 to 400 mg celecoxib.

10. The prophylaxis composition of claim 5, wherein said composition comprises about 10 to 20 mg piroxicam.

11. The prophylaxis composition of claim 1, wherein said composition is in the form of a tablet, capsule, or liquid.

12. The prophylaxis composition of claim 1, wherein said composition also provides cardiac protection.

13. The prophylaxis composition of claim 12, wherein said composition comprises:
(i) at least one form of elemental magnesium; and
(ii) a therapeutically effective concentration of acetylsalicylic acid;
wherein said prophylaxis composition is a single dosage form.

14. A method of preventing a migraine in a patient in need thereof comprising administering a migraine prophylaxis composition, wherein the prophylaxis composition is a single dosage form comprising:
(i) at least one form of elemental magnesium; and
(ii) at least one NSAID.

15. The method of claim 14, wherein said prophylaxis composition is administered on a daily, ongoing basis.

16. The method of claim 14, wherein said prophylaxis composition is administered once a day.

17. The method of claim 14, wherein said prophylaxis composition is administered twice a day.

18. The method of claim 14, wherein said prophylaxis composition is administered orally.

19. The method of claim 14, wherein said single dosage form is a tablet, a capsule or liquid.

20. A method of providing cardiac protection and preventing cardiovascular disease in a patient in need thereof comprising administering a prophylaxis composition, wherein the prophylaxis composition is a single dosage form comprising:
(i) at least one form of elemental magnesium; and
(ii) a therapeutically effective concentration of acetylsalicylic acid.

21. The method of claim 20, wherein said prophylaxis composition is administered on a daily, ongoing basis.

22. The method of claim 20, wherein said prophylaxis composition is administered once a day.

23. The method of claim 20, wherein said prophylaxis composition is administered twice a day.

24. The method of claim 20, wherein said prophylaxis composition is administered orally.

25. The method of claim 20, wherein said single dosage form is a tablet, a capsule or liquid.
